(19) United States
(12) Patent Application Publication
(10) Pub. No.: US 2026/0167994 A1
(43) Pub. Date: Jun. 18, 2026

(54) METHOD FOR PREPARING AN AQUEOUS SOLUTION CONTAINING AN ALKALI SALT OF GLYCOLIC ACID AND LACTIC ACID

(71) Applicant: ANNIKKI GMBH, Raaba-Grambach (AT)

(72) Inventors: Nicole STAUNIG, Raaba-Grambach (AT); Maria DUPONT, Raaba-Grambach (AT)

(21) Appl. No.: 19/122,704

(22) PCT Filed: Oct. 20, 2023
(86) PCT No.: PCT/EP2023/079260

§ 371 Date: Apr. 18, 2025

(30) Foreign Application Priority Data
Oct. 20, 2022 (EP) 22202789.8

(51) Int. Cl. C12P 7/56; C12N 9/02; C12N 9/04; C12N 9/88; C12P 7/42

(52) U.S. Cl. CPC C12P 7/56; C12N 9/0006; C12N 9/0008; C12N 9/88; C12P 7/42; C12Y 101/01027; C12Y 102/01021; C12Y 401/02; C12Y 402/01025

(57) ABSTRACT

The invention relates to a method for preparing an aqueous solution containing an alkali salt of glycolic acid and lactic acid, by treating an alkali salt of xylonic acid and/or arabonic acid in an aqueous solution in vitro with an enzyme system comprising a dehydratase, an aldolase, a glycolaldehyde dehydrogenase, a lactate dehydrogenase, and NAD⁺ as a cofactor, after which the enzyme system is separated. (Figure)

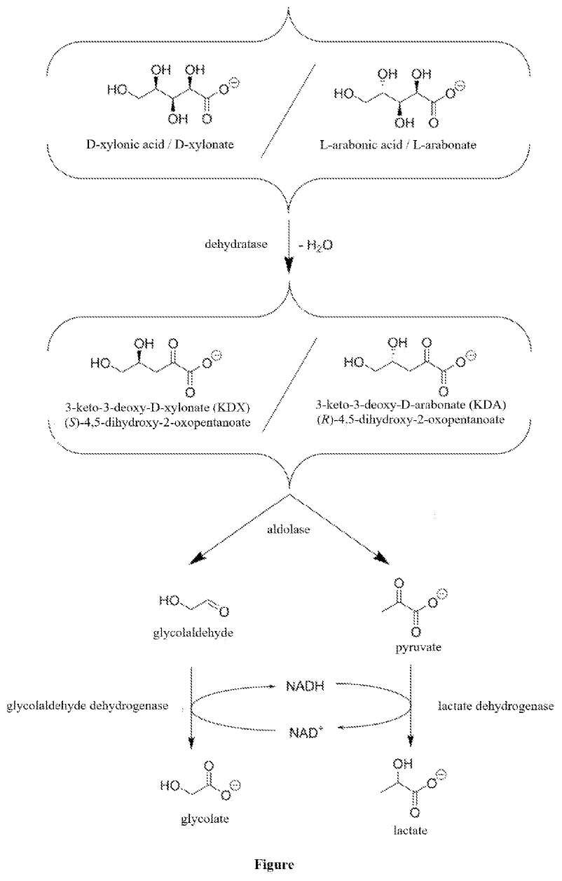

The invention relates to a method for preparing an aqueous solution containing an alkali salt of glycolic acid and lactic acid. Such solutions are used in the cosmetics industry as a base for skin peeling products.

The invention involves a carbon economic enzymatic process to generate chemicals from renewable carbon sources. Specifically, it concerns the atom economic production of alkali glycolate and alkali lactate through an enzymatic process, wherein D-xylonate and L-arabonate, the C₁ oxidation products of the pentoses D-xylose and L-arabinose, are converted into the C₂ building block glycolic acid (glycolate) and the C₃ building block lactic acid (lactate).

In this document, D-xylose is used interchangeably with xylose, L-arabinose interchangeably with arabinose, L-arabonate interchangeably with arabonate and arabonic acid, D-xylonate interchangeably with xylonate and xylonic acid, glycolate interchangeably with glycolic acid, lactate interchangeably with lactic acid, as well as glucose interchangeably with D-glucose.

### BACKGROUND OF THE INVENTION

Although glycolic acid occurs naturally, for instance in sugarcane and sugar beet, its synthesis currently is realised predominantly from fossil fuels due to the low concentrations of the substance in these renewable raw materials. As in the synthetically produced glycolic acid there are often present residues of formaldehyde, which is particularly problematic when used in cosmetic products (e.g., in skin scrubs (Sharad, 2013)), there is a need for alternative synthesis methods.

Chemo-enzymatic methods for the production of glycolic acid comprise the conversion of glycolonitrile, synthesised from formaldehyde and hydrogen cyanide, to glycolic acid via nitrilases (Panova et al., 2008; Ben-Bassat et al., 2008). This method cannot be considered as being sustainable due to the toxic, energy-intensive chemicals used, which are generally derived from fossil sources. Another method is the conversion of ethylene glycol to glycolic acid by means of microbial whole-cell biocatalysts (Gao et al., 2014). In this second instance, too, the starting material is also typically produced through energy-intensive chemical processes. A chemical method for producing glycolic acid that utilises a renewable raw material as a starting material is the conversion of glyoxal derived from bio-oil to glycolic acid in the presence of zeolite as a catalyst (Dapsens et al., 2014). Bio-oil is obtained through the pyrolysis of biomass—a high-energy process that destroys the original composition of the biomass, leaving the synthetic potential of the raw material untapped.

In order to develop sustainable production methods for chemicals from biomass, less energy-intensive methods are being required that make use of the existing variety of biomass materials. Apart from efficient, mild methods for the depolymerisation of renewable raw materials into shorter-chain sugars, there are further needed efficient technologies for further converting these carbohydrate intermediates into chemical products. A particular challenge in this context is to efficiently and as completely as possible convert the mixtures of substances generated after depolymerisation into valuable material streams. In general, straw, wood, and other plant-based raw materials contain hemicellulose and cellulose, from which monomeric sugars may be released in varying compositions, primarily the pentoses xylose and arabinose, the hexoses glucose, mannose, and galactose, as well as acids derived from these sugars (e.g., glucuronic acid). Methods for the efficient separation of C₆ and C₅ sugars allow these types of material streams to be treated separately (e.g., WO 2011/014894). For instance, maize husks contain, in relation to total sugars, 10% arabinose, 16% xylose, 64% glucose, 4% galactose, and 2% mannose (Hromádková & Ebringerová, 1995), while wheat straw contains 5% arabinose, 30% xylose, 56% glucose, 1% galactose, and 2% mannose (Collins et al., 2014). These percentages demonstrate that the ratios of arabinose to xylose differ significantly between these two biomass types (maize husks: 1:1.6, wheat straw: 1:6).

Currently, biotechnological efforts primarily focus on methods aimed at the conversion of biomass into chemical products by means of fermentations, i.e., the transformation of substrates during the growth of microbes in a reactor. These fermentative whole-cell processes, despite all technical progress in the field, such as metabolic engineering and heterologous pathway expression, are limited by the physiological constraints of cellular production (tolerance to solvents, temperature, substance transport, as well as to high substrate and product concentrations, but also by by-products from other metabolic pathways within the cell) (Claassens et al., 2019). These intracellular processes, and in particular the energy demand for the metabolic (often CO₂ emitting) steps, lead to long processing times and a significantly lower carbon yield than theoretically possible. Furthermore, these processes generally utilise biosynthetic metabolic pathways that release CO₂ from the substrate, such that even the theoretical carbon yield in the product is only a fraction of the carbon used: depending on the metabolic pathway, 1 to 4 carbons are lost from C₆ sugars, and 1 to 3 from C₅ sugars. Typical key data for such processes are summarised in the literature (Salusjärvi et al., 2019).

For example, the fermentative production of glycolic acid from glucose via the glyoxylate shunt using metabolic engineering in recombinant E. coli strains is known (WO 2007/141316A2, WO 2007/140816A1, WO 2010/108909A1, WO 2011/036213A2). In one case, in a fermentation process lasting 93 hours there was achieved the fermentative production of 31.3 g/l glycolic acid in the fermentation supernatant, with yields of 0.22 g glycolic acid per gram of glucose (WO 2007/141316A2). WO 2011/036213A2 then describes a further optimised fermentation process of the system, obtaining 53.9 g/l glycolic acid within 40 hours through adapted process management, with a yield of 0.36 g glycolic acid per gram of glucose.

Moreover, the fermentative production of glycolic acid from pentoses, e.g., through the combination of the ribulose-1-phosphate pathway and the glyoxylate shunt, is described in the literature (Pereira et al., 2016). In this process, a yield of 0.62 g glycolic acid per gram of xylose is achieved through metabolic engineering of an E. coli strain. The glycolic acid endpoint concentration after 85 hours is 41 g/l in the fermentation broth, with a yield of 61% of the theoretical (1.22 mol/mol). In general, the upper limit for fermentative approaches to glycolic acid production currently amounts to 65.5 g/l glycolic acid endpoint concentration and 90% of the theoretical yield (where 100% theoretical yield implies the loss of one carbon atom per sugar molecule) when pure glucose is used as the substrate (Deng et al., 2018; Salusjärvi et al., 2019). For pure xylose, the achieved values are similar (44 g/l glycolic acid endpoint concentration, 87% of the theoretical yield (Pereira et al., 2016; Salusjärvi et al., 2019)). However, the achievable glycolic acid concentrations in sugar mixtures (xylose+glucose) are an order of magnitude lower (Alkim et al., 2016; Salusjärvi et al., 2019). This is also the case in analogous systems that realise the fermentative conversion of sugars to ethylene glycol (Pereira et al., 2016; Salusjärvi et al., 2017; Uranukul et al., 2018; Salusjärvi et al., 2019).

In summary, it can be said that fermentative systems in general accept the loss of at least one carbon per sugar monomer and achieve only low product endpoint concentrations in sugar mixtures.

Cell-free conversions of metabolites were first demonstrated by Eduard Buchner in 1897, who converted glucose into ethanol using a cell lysate from Saccharomyces cerevisiae (Buchner, 1897). In 1985, Welch and Scopes presented a cell-free system for the ethanol production (Welch & Scopes, 1985), which was, however, technologically unusable due to a lack of specificity.

Since then, a number of further processes have been described for the production of chemicals from purified enzymes (enzyme isolates). For instance, alcohol dehydrogenases have been used for the production of high-quality chiral alcohols, wherein the cofactor NAD is being regenerated, for example, by the addition of glucose and glucose dehydrogenase (Goldberg et al., 2007). In general, glucose or formate dehydrogenases (GDHs or FDHs) and alcohol dehydrogenases (ADHs) are used for cofactor recycling (Schrittwieser et al., 2018).

In recent years, interest has shifted towards processes that use highly selective conversions by means of enzyme cascade reactions to obtain the target chemical in a single step (one-pot). EP2700714A1, U.S. Pat. No. 8,859,247B2, and EP2204453B1 describe a portfolio of cascade reactions, in which glucose is converted to two molecules of pyruvate through a cascade of five enzymatic conversions. Starting from glucose, the cascade follows the non-phosphorylating Entner-Doudoroff pathway to D-glyceraldehyde and pyruvate (sugar oxidation, dehydration, and aldol cleavage). D-glyceraldehyde is then further oxidised to D-glycerate and subsequently dehydrated to pyruvate. This then serves as a platform for further transformations into a range of amino acids and alcohols. The enzymes of the cascade thus comprise a glucose dehydrogenase, a promiscuous dihydroxy acid dehydratase (which catalyses the dehydrations of gluconate and D-glycerate), a 2-keto-3-deoxygluconate aldolase, and an aldehyde dehydrogenase. By using a similarly promiscuous dehydrogenase, which accepts both glucose and D-glyceraldehyde as substrates, the system may be minimised to three enzymes.

A similar cascade exists for the pentose xylose, known as the Dahms pathway (Dahms, 1974). Here, xylose is first oxidised to 1,4-xylonolactone, which is opened to xylonate under the influence of a lactonase. A dehydratase converts xylonate to 2-keto-3-deoxy-xylonate, which is then cleaved by an aldolase into pyruvate and glycolaldehyde. Through this pathway, arabinose can also be converted in organisms such as Sulfolobus solfataricus, thanks to the promiscuous enzymes of the pathway (Kopp et al., 2020).

Pyruvate is a central intermediate in cellular metabolism and serves, for example, as a precursor to the amino acid alanine or to acetyl-CoA, which is involved in the citric acid cycle. Through the reduction of pyruvate, lactic acid or lactate, respectively, is produced, which has a wide range of applications. Lactic acid is used, for instance, as an acidifier in the food industry, as a descaling agent, pH regulator, or cleaning agent in the chemical industry, as an additive in anti-acne creams or moisturisers in the cosmetics industry, and as a precursor for acrylic acid or ethyl lactate (Wee et al., 2006). Furthermore, lactic acid (like glycolic acid) may also be used for skin peeling (chemical peeling) (Smith, 1996). The bifunctional lactic acid may also be polymerised, yielding polylactide (PLA), a biodegradable and biocompatible plastic with applications in the packaging industry, textile industry, electronics, and medicine field (Balla et al., 2021).

Boer et al. presented an in vitro variant of the Dahms pathway with purified enzyme isolates, where xylose or xylonolactone, respectively, (1 mM substrate concentration) is converted to ethylene glycol (reduction of glycolaldehyde), glycolic acid (oxidation of glycolaldehyde), or lactate (reduction of pyruvate). The main focus of the study was the application of a spectrophotometric assay to evaluate the efficiency of the cascade, based on the formation or consumption of NADH. For this purpose, 2 mM NAD as an external cofactor was added. Furthermore, the study also investigated the role of lactonase (from Caulobacter crescentus) in the Dahms pathway. The data suggest that the spontaneous ring-opening of xylonolactone is rate-limiting, particularly at pH 7. Only with the addition of lactonase is the overall kinetics of the cascade significantly accelerated, although this may also be achieved by increasing the pH (Boer et al., 2019).

From WO 2014/162063A1 there is known to obtain glycolic acid alongside lactic acid via a fermentative way in a metabolically engineered eukaryotic system (S. cerevisiae) from the pentose xylose. However, the achieved product concentrations are far below the theoretical yield. For instance, in the cofermentation of glucose (10 g/l) and xylose (20 g/l) over a fermentation duration of 2 days, only 0.927 g/l of lactic acid and 0.696 g/l of glycolic acid are produced, with concentrations from pure sugar fermentations being even lower.

The present invention addresses this issue with the aim of providing a method for preparing an aqueous solution containing an alkali salt of both glycolic acid and lactic acid, which exhibits high yields and is operable at higher concentrations.

### DETAILED DESCRIPTION OF THE INVENTION

This objective is achieved by a method, in which an alkali salt of xylonic acid and/or arabonic acid is treated in an aqueous solution in vitro with an enzyme system that contains a dehydratase, an aldolase, a glycolaldehyde dehydrogenase, a lactate dehydrogenase, and NAD⁺ as a cofactor, after which the enzyme system is separated.

Surprisingly, it has been shown that the objects set forth in the invention may be attained when the conversion is not carried out fermentatively, but rather the enzymes are present as such in the aqueous solution, thus operating in vitro.

The term “enzyme system” refers to the entirety of the four enzymes, namely the dehydratase, the aldolase, the glycolaldehyde dehydrogenase, the lactate dehydrogenase, as well as NAD⁺ as the cofactor. These four enzymes together with the cofactor as such (enzyme isolates) may, for example, be added to the aqueous solution. However, it is also possible to suspend cells that express the specified enzymes in the aqueous solution. In this case, the term “enzyme system” is to be understood as a cell suspension. Further, the term “enzyme system” is to be understood as a homogenate that may be obtained from the cell suspension, for example, by exposing the cells to ultrasound to cause them to burst. Further, the term “enzyme system” is also to be understood as lysates that are obtained from the homogenates by separating the solid cellular components. Finally, the term “enzyme system” is also to be understood as enzymes that are present immobilised in or on a matrix.

A major advantage of the method according to the invention is that the conversion may also be carried out under non-physiological conditions, such as high substrate concentrations, which are concentrations, at which a fermentative process cannot operate.

Another major advantage of the method according to the invention is that it may be carried out at low NAD⁺ concentrations because this is intrinsically regenerated during this method. The intrinsic regeneration is possible because the formation of glycolate from glycolaldehyde is an oxidation that consumes NAD⁺ while generating NADH, whereas the formation of lactate from pyruvate is a reduction that consumes NADH, thereby regenerating NAD⁺. This intrinsic regeneration is a decisive advantage due to the high cost of NAD⁺ and facilitates the large-scale implementation of the process.

The reaction scheme is illustrated in the accompanying figure, which also shows the intrinsic regeneration. Oxidation and reduction may be started by NAD⁺, but equally by NADH, such that for the purposes of the present patent claims and description, NAD⁺ also refers to the corresponding NADH.

In a further preferred variant of the method according to the invention, the enzymes forming the enzyme system are present in a suspension, homogenate, and/or lysate of the corresponding cells forming them. It has been demonstrated that, when using suspensions, lysates, and/or homogenates, the cofactor for the dehydrogenases does not need to be added separately, as the small amounts of cofactor present in the suspension, lysate, and/or homogenate are already sufficient due to the intrinsic regeneration of NAD⁺ to provide for the final step of oxidation and reduction.

Through the intrinsic cofactor regeneration, the system is thus able to operate without the addition of an organic co-substrate, which would impair energy and carbon efficiency and complicate processing. Consequently, the method according to the invention expands the core reactions of the Dahms pathway—namely dehydration and aldol cleavage—by integrating a closed redox system that oxidises glycolaldehyde to glycolic acid and reduces pyruvate to lactate, all using the same pair of cofactors (NAD⁺/NADH).

In this context, suspension refers to a suspension of resting cells. These cells are harvested after cultivation (separated from the nutrient medium) and suspended in a suitable buffer system. In contrast to fermentative methods, which also work with whole cells, the resting cells can no longer grow due to the removal of carbon sources and nutrients, but rather serve solely for the conversion of substrates (Lin & Tao, 2017). Homogenate in this context refers to a physically and/or chemically treated suspension (e.g., treated by means of pressure, lysozyme, or ultrasound), wherein the cellular components are released from the cells. A lysate is obtained when the insoluble cellular components of the homogenate are removed, for example, through filtration or centrifugation (see Production of the Enzymes for details).

According to a preferred embodiment of the method according to the invention, there is used a potassium salt as the alkali salt.

In further preferred embodiments of the method according to the invention, the concentration of the alkali salt of xylonic acid and/or arabonic acid in the aqueous solution is between 50 and 300 g/l, 100 and 200 g/l, and ultimately 150 and 250 g/l.

Additionally, it is preferred that the method according to the invention is carried out at a temperature of between 2° and 50° C., between 25 and 40° C., and even more preferably between 3° and 40° C.

The in particular preferred pH range of the reaction lies between 7.5 and 8.5.

According to another preferred aspect of the invention, all reaction steps of the method, as well as the regeneration of the cofactors, take place in a single reaction vessel (one-pot reaction), thereby avoiding the costly isolation of intermediates. In this embodiment, all enzymes are used at the beginning of the reaction.

The present invention is further based on the surprising finding that the yield of glycolate, for example, is higher when lactate is simultaneously formed by the use of lactate dehydrogenase, compared to the sole production of glycolate (and vice versa, see Example 5 below).

The conversion of arabonate and/or xylonate via 4,5-dihydroxy-2-oxopentanoate ((R)-4,5-dihydroxy-2-oxopentanoate=2-keto-3-deoxy-arabonate (KDA) and/or (S)-4,5-dihydroxy-2-oxopentanoate=2-keto-3-deoxy-xylonate (KDX)) to pyruvate and glycolaldehyde, hence, comprises a dehydratase and an aldolase.

The dehydratase used in the method may be derived from one of the following groups: EC 4.2.1.5 (arabonate dehydratase), 4.2.1.6 (galactonate dehydratase), 4.2.1.7 (altronate dehydratase), 4.2.1.8 (mannonate dehydratase), 4.2.1.9 (dihydroxy acid dehydratase), 4.2.1.25 (L-arabonate dehydratase), 4.2.1.39 (gluconate dehydratase), 4.2.1.40 (glucuronate dehydratase), 4.2.1.42 (galactarate dehydratase), 4.2.1.67 (D-fuconate dehydratase), 4.2.1.68 (L-fuconate dehydratase), 4.2.1.82 (xylonate dehydratase), 4.2.1.140 (gluconate/galactonate dehydratase), 4.2.1.146 (L-galactonate dehydratase), 4.2.1.156 (L-talarate dehydratase), 4.2.1.158 (galactarate dehydratase, D-threo-forming), and 4.2.1.176 (L-lyxonate dehydratase), wherein the group 4.2.1.25 (L-arabonate dehydratase) is particularly preferred.

The aldolase used in the method may be derived from one of the following groups: EC 4.1.2.18 (2-dehydro-3-deoxy-L-pentonate aldolase), 4.1.2.20 (2-dehydro-3-deoxyglucuronate aldolase), 4.1.2.21 (2-dehydro-3-deoxy-6-phosphogalactonate aldolase), 4.1.2.28 (2-dehydro-3-deoxy-D-pentonate aldolase), 4.1.2.29 (5-dehydro-2-deoxyphosphogluconate aldolase), 4.1.2.51 (2-dehydro-3-deoxy-D-gluconate aldolase), 4.1.2.52 (4-hydroxy-2-oxoheptanedioate aldolase), 4.1.2.53 (2-keto-3-deoxy-L-rhamnonate aldolase), 4.1.2.54 (L-threo-3-deoxy-hexulose aldolase), 4.1.2.55 (2-dehydro-3-deoxy-phosphogluconate/2-dehydro-3-deoxy-6-phosphogalactonate aldolase), and 4.1.3.39 (4-hydroxy-2-oxovalerate aldolase), wherein the group 4.1.2.55 (2-dehydro-3-deoxy-phosphogluconate/2-dehydro-3-deoxy-6-phosphogalactonate aldolase) is particularly preferred.

The dehydrogenase used for the reduction of pyruvate to lactate is preferably derived from the group EC 1.1.1.27 (L-lactate dehydrogenase).

The dehydrogenase used for the oxidation of glycolaldehyde to glycolic acid is preferably derived from the group EC 1.2.1.21 (glycolaldehyde dehydrogenase).

The enzymatic strategy presented herein minimises the number of enzymes, thus enabling a highly efficient and cost-effective/costly competitive bioproduction process.

The following examples describe preferred embodiments of the invention in greater detail.

### Materials

2-Keto-3-deoxyxylose lithium salt, sodium pyruvate, sodium L-lactate, and glycolaldehyde dimer, IPTG (isopropyl β-D-thiogalactopyranoside), and HEPES (2-(4-(2-hydroxyethyl)-1-piperazinyl) ethanesulfonic acid) were obtained from Sigma-Aldrich, magnesium chloride hexahydrate, potassium dihydrogen phosphate, dipotassium hydrogen phosphate, NAD⁺, NADH disodium salt, and sodium dodecyl sulfate (SDS) were acquired from Carl Roth, lysozyme and methanol were acquired from PanReac AppliChem (ITW Reagents), triethanolamine was obtained from Chem-Lab NV, and sodium glycolate was acquired from Alfa Aesar. Potassium L-arabonate and potassium D-xylonate were produced according to the literature method (Moore & Link, 1940) from the corresponding sugars.

### Production of the Enzymes

For the recombinant enzyme production in an Escherichia coli strain, the gene to be expressed was first amplified in a PCR using genomic DNA or its synthetic equivalent adapted to the codon usage of E. coli as a template, along with specific oligonucleotides that additionally carry recognition sequences for restriction endonucleases, and then it was isolated from the reaction mixture. Following digestion of the nucleic acids with the restriction enzymes SphI and HindIII, the gene fragment coding for the target enzyme was ligated into the SphI and HindIII-cut backbone of the expression vector pQE70-Kan. The ligation product was transformed into chemically competent E. coli cells Top10F, and the resulting colonies were used for plasmid isolation and restriction analysis.

The result of the cloning step was verified by means of restriction enzyme digestion and DNA sequencing. The resulting construct carries the target gene under the IPTG-inducible T5 promoter.

For the overexpression of the enzyme in E. coli, the resulting expression plasmid was transformed into the competent expression cells RB791. After 24 hours of incubation at 37° C., the resulting colonies were inoculated into LB medium for expression tests.

The following day, expression cultures were inoculated at an optical density OD₅₅₀ of 0.02 and shaken at 37° C. until an OD₅₅₀ of 0.3 was reached. Subsequently, the temperature was lowered to 25° C., and the cultures were induced with 0.1 mM IPTG upon reaching an OD₅₅₀ of 0.5. After 22 hours, the cultures were harvested (separated from the medium by means of centrifugation into a cell pellet) and analysed for the expression of the recombinant enzyme using SDS gel electrophoresis and an activity assay (utilised in a use-test or optical-enzymatic assay).

### Preparation of Cell Suspensions

In order to prepare cell suspensions, the cell pellets produced according to the method above were weighed into a suitable vessel, and a buffer was added (see Table 1 for the buffer systems used) while stirring in an ice bath. The biomass mass fraction typically amounted to 20%, with the remainder consisting of the buffer.

### Preparation of Homogenates Via Disruption Via Sonification

Lysozyme was added to the cell suspension prepared above at a concentration of 0.5 mg/ml. A Branson Sonifier 450 was used for cell disruption. The suspension was transferred into a 5 ml Eppendorf vial. The metal tip of the apparatus was then immersed in the suspension, after which the suspension was treated with three cycles of 15 ultrasonic pulses each (device settings: Timer=15; Duty Cycle=50; Output Control=3-5). In this manner, the homogenate was obtained as a mixture of disrupted cells and buffer.

### Preparation of Lysates by Centrifugation

The homogenate prepared above was centrifuged for 10 minutes at 4° C. and 16,000 rpm (Eppendorf Centrifuge 5417R) to separate the insoluble cell fragments and obtain the lysate.

TABLE 1
Donor organisms and disruption conditions for the enzymes used in the examples.

| enzyme type (EC | catalysed | | disruption | |
|---|---|---|---|---|
| class) | reaction(s) | donor organism | conditions | literature |
| dehydratase (EC | xylonate/ | Azotobacter | Sonifier; 20% | (Setubal et al., 2009); |
| 4.2.1.25) | arabonate → KDX/ | vinelandii DJ | biomass in 100 mM | (NLM Protein |
| | KDA | | TEA-HCl, pH 7 | database: |
| | | | | ACO81022.1) |
| aldolase (EC | KDX/KDA → | Sulfolobus | Sonifier; 20% | (Wolterink-van Loo et |
| 4.1.2.55) | glycolaldehyde + | acidocaldarius | biomass in 100 mM | al., 2009) |
| | pyruvate | | TEA-HCl, pH 7 | |
| glycolaldehyde | glycolaldehyde → | Escherichia coli | Sonifier; 20% | (Hidalgo et al., 1991) |
| dehydrogenase | glycolic acid | | biomass in 100 mM | |
| (EC 1.2.1.21) | | | KPP, pH 7 | |
| L-lactate | pyruvate→ I-lactate | Oryctolagus | Sonifier; 50% | (Zheng et al., 2004) |
| dehydrogenase | | cuniculi (rabbit | biomass in 50 mM | |
| (EC 1.1.1.27) | | muscle) | HEPES, pH 7.5 | |

### Analytical Methods

### High Performance Anion Exchange Chromatography

Substrate conversions and product concentrations were determined using HPAEC (High Performance Anion Exchange Chromatography). A Dionex ICS6000 system equipped with an AS-AP autosampler was employed for this purpose. The measurement of organic acids or their anions (xylonate, arabonate, 2-keto-3-deoxy-xylonate, 2-keto-3-deoxy-arabonate, lactate, and pyruvate), respectively, was carried out by means of conductivity detection (CD) coupled with a Dionex AERS 500 electrolytically regenerated suppressor in external water mode. A Dionex IonPac AS11-HC-4 μm column, together with an appropriate precolumn as well as an NaOH gradient, was utilised for the separation of the analytes. The mobile phase was additionally pre-treated with a Dionex ATC Anion Trap Column.

### High Performance Liquid Chromatography

For the quantification of glycolaldehyde and glycolic acid, HPLC (High Performance Liquid Chromatography) was used. Detection was performed using a refractive index detector. A Phenomenex Rezex ROA-Organic Acid H+ (8%) column, together with a corresponding precolumn, was used for the measurement and eluted isocratically with 1 mM sulphuric acid.

### Determination of Enzyme Activities (Optical-Enzymatic Assay)

Enzyme activities in the homogenates or lysates, respectively, were determined using a Shimadzu UV-1900 spectrophotometer. The formation or consumption, respectively, of NADH was monitored at a wavelength of 340 nm by tracking the changes in absorption. Measurements were conducted with 0.2 mM cofactor (NAD⁺ or NADH). For this purpose, 20 μl of a 10 mM stock solution of the cofactor was placed in a cuvette (Greiner bio-one Semi-Micro Cuvette made of polystyrene), and the desired pH was adjusted using 100 mM TEA-HCl buffer (870 μl). Following temperature equilibration of the cuvette, 10 μl of homogenate or lysate (diluted or undiluted), respectively, and 100 μl of substrate solution were added, and measurement was started immediately thereafter. Measurements were routinely performed at 25° C. The enzyme activity of the lysate may be determined in U/ml (based on the volume of the lysate) or U/g (based on the biomass used for production) using the extinction coefficient of NADH at 340 nm (¿=6220 L mol⁻¹ cm⁻¹). 1 U corresponds to 1 μmol of substrate conversion per minute (1 U=1 μmol/min=1.67×10⁻⁸ kat).

### General Information on the Processing of the Reaction Solution

The aqueous solution containing glycolate and lactate, prepared according to the invention, is obtained by removing cellular components through denaturation (e.g., via heat treatment) followed by filtration or centrifugation after the reaction has concluded. In order to remove smaller cellular components, membrane filtration may also be performed. The thus obtained filtrate may then be concentrated, for example, using a rotary evaporator.

The following examples describe preferred variants of the method according to the invention in greater detail. The suspensions, homogenates, and lysates used in these examples were prepared according to the methods described above.

### Example 1

Treatment of Xylonate/Arabonate Mixtures in Different Compositions (9:1 and 2:1 m/m) with the Enzyme System

In two 2 ml Eppendorf vials (vial 1 and vial 2), the following components were prepared: 100 μl of a 2000 mM TEA-HCl buffer (pH 8.5), 200 μl of deionised water, 40 μl of dehydratase lysate, 40 μl of aldolase lysate, 2 U of glycolaldehyde dehydrogenase lysate, and 2 U of lactate dehydrogenase lysate. The reactions were started by the addition of 100 μl of a solution of potassium xylonate and potassium arabonate to each of the Eppendorf vials. The solution for vial 1 contained 9 parts potassium xylonate (225.7 g/l) and 1 part potassium arabonate (25.0 g/l), while the solution for vial 2 contained 2 parts potassium xylonate (166.3 g/l) and 1 part potassium arabonate (84.0 g/l). The batches were incubated for a total of 48 hours at 30° C. with continuous shaking (1200 rpm, Eppendorf Thermomixer). The total volume of each batch was 500 μl.

For processing, 40 μl of a batch was combined with 160 μl of ultrapure water and 200 μl of MeOH and incubated at 60° C. for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | | conversion |
|---|---|---|---|---|
| | | concentration | mass | after 48 h |
| | | [g/l] | [mg] | [%] |
| vial 1 - xylonate/arabonate 9:1 | | | | |
| substrates | potassium xylonate | 45.1 | 22.6 | 55 * |
| (t = 0 h) | potassium | 5.0 | 2.5 | |
| | arabonate | | | |
| products | glycolate ** | 9.5 | 4.7 | 51 |
| (t = 48 h) | lactate ** | 9.7 | 4.8 | 44 |
| vial 2 - xylonate/arabonate 2:1 | | | | |
| substrates | potassium xylonate | 33.3 | 16.6 | 63 * |
| (t = 0 h) | potassium | 16.8 | 8.4 | |
| | arabonate | | | |
| products | glycolate ** | 10.0 | 5.0 | 54 |
| (t = 48 h) | lactate ** | 9.9 | 5.0 | 45 |

* The substrate conversion may only be specified as a sum parameter due to peak overlap in HPAEC.
** The concentrations and masses of the products glycolate and lactate, as well as their conversions, were calculated for the respective free acids.

The tables above indicate that the enzyme system may process different mix ratios of xylonate and arabonate with similar efficiency. This is relevant, as the corresponding pentoses (xylose and arabinose) may be released from biomass, where they occur in varying proportions. Therefore, these may be oxidised without prior separation, and the resulting mixtures of sugar acids may be directly converted to glycolate and lactate by the enzyme system.

### Example 2

Influence of Various Enzyme Formulations (Suspension, Homogenate, or Lysate) on the Treatment of a Xylonate/Arabonate (9:1 w/w) Mixture with the Enzyme System

In a 2 ml Eppendorf vial, the following components were prepared: 100 μl of a 2000 mM TEA-HCl buffer (pH 8.5), 200 μl of deionised water, 40 μl of a dehydratase formulation (see subsequent tables for details), 40 μl of an aldolase formulation (see subsequent tables for details), 2 U of a glycolaldehyde dehydrogenase formulation, and 2 U of lactate dehydrogenase lysate. The reaction was started by the addition of 100 μl of a solution containing 9 parts potassium xylonate (225.7 g/l) and 1 part potassium arabonate (25.0 g/l). The batch was incubated for a total of 48 hours at 30° C. with continuous shaking (1200 rpm, Eppendorf Thermomixer). The total volume was 500 μl.

For processing, 40 μl of the batch was combined with 160 μl of ultrapure water and 200 μl of MeOH and incubated at 60° C. for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | | conversion |
|---|---|---|---|---|
| | | concentration | mass | after 48 h |
| | | [g/l] | [mg] | [%] |
| dehydratase as homogenate; | | | | |
| aldolase as suspension; | | | | |
| glycolaldehyde-DH as | | | | |
| homogenate; L-lacDH as lysate | | | | |
| substrates | potassium xylonate | 45.1 | 22.6 | 44 * |
| (t = 0 h) | potassium | 5.0 | 2.5 | |
| | arabonate | | | |
| products | glycolate ** | 6.5 | 3.2 | 35 |
| (t = 48 h) | lactate ** | 5.3 | 2.7 | 24 |
| dehydratase as lysate; | | | | |
| aldolase as suspension; | | | | |
| glycolaldehyde-DH as | | | | |
| lysate; L-laDH as lysate | | | | |
| substrates | potassium xylonate | 45.1 | 22.6 | 47 * |
| (t = 0 h) | potassium | 5.0 | 2.5 | |
| | arabonate | | | |
| products | glycolate ** | 7.6 | 3.8 | 40 |
| (t = 48 h) | lactate ** | 6.6 | 3.3 | 30 |
| dehydratase as homogenate; | | | | |
| aldolase as homogenate; | | | | |
| glycolaldehyde-DH as | | | | |
| homogenate; L-lacDH as lysate | | | | |
| substrates | potassium xylonate | 45.1 | 22.6 | 52 * |
| (t = 0 h) | potassium | 5.0 | 2.5 | |
| | arabonate | | | |
| products | glycolate ** | 7.5 | 3.8 | 40 |
| (t = 48 h) | lactate ** | 6.6 | 3.3 | 30 |

* The substrate conversion may only be specified as a sum parameter due to peak overlap in HPAEC.
** The concentrations and masses of the products glycolate and lactate, as well as their conversions, were calculated for the respective free acids.

The tables above indicate that various enzyme formulations (suspensions, homogenates, and lysates) may be used for the enzyme system. The highest conversion is achieved when all enzymes are added in the form of lysates (see vial 1 of Example 1).

### Example 3

Influence of NAD⁺ Addition on the Treatment of a Xylonate/Arabonate (9:1 w/w) Mixture with the Enzyme System

In a 2 ml Eppendorf vial (vial 1), the following components were prepared: 100 μl of a 2000 mM TEA-HCl buffer (pH 8.5), 200 μl of deionised water, 40 μl of dehydratase lysate, 40 μl of aldolase lysate, 2 U of glycolaldehyde dehydrogenase lysate, and 2 U of lactate dehydrogenase lysate. In a second 2 ml Eppendorf vial (vial 2), a similar mixture was prepared, but with the addition of 10 μl of a 10 mM NAD⁺ solution and with 190 instead of 200 μl of deionised water. The reactions were started by the addition of 100 μl of a solution containing 9 parts potassium xylonate (225.7 g/l) and 1 part potassium arabonate (25.0 g/l) to both Eppendorf vials. The batches were incubated for a total of 48 hours at 30° C. with continuous shaking (1200 rpm, Eppendorf Thermomixer). The total volume of each batch was 500 μl.

For processing, 40 μl of a batch was combined with 160 μl of ultrapure water and 200 μl of MeOH and incubated at 60° C. for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | | conversion |
|---|---|---|---|---|
| | | concentration | mass | after 48 h |
| | | [g/l] | [mg] | [%] |
| vial 1 - no addition of cofactor | | | | |
| substrates | potassium xylonate | 45.1 | 22.6 | 55 * |
| (t = 0 h) | Potassium | 5.0 | 2.5 | |
| | Arabonate | | | |
| products | glycolate ** | 9.5 | 4.7 | 51 |
| (t = 48 h) | lactate ** | 9.7 | 4.8 | 44 |
| vial 2 - addition of cofactor (0.2 | | | | |
| mm NAD⁺ final) | | | | |
| substrates | potassium xylonate | 45.1 | 22.6 | 57 * |
| (t = 0 h) | potassium | 5.0 | 2.5 | |
| | arabonate | | | |
| products | glycolate ** | 9.0 | 4.5 | 48 |
| (t = 48 h) | lactate ** | 8.5 | 4.2 | 38 |

* The conversion of the substrates may only be specified as a sum parameter due to peak overlap in HPAEC.
** The concentrations and masses of the products glycolate and lactate, as well as their conversions, were calculated for the respective free acids.

The tables above indicate that the addition of cofactor (0.2 mM NAD⁺) does not confer any advantages regarding conversion under these conditions.

### Example 4

Treatment of a Xylonate/Arabonate (9:1 w/w) Mixture (Substrate Concentration: 100 g/l) with the Enzyme System

In a 2 ml Eppendorf vial, the following components were prepared: 150 μl of a 2000 mM TEA-HCl buffer (pH 8.5), 90 μl of deionised water, 60 μl of dehydratase lysate, 60 μl of aldolase lysate, 4 U of glycolaldehyde dehydrogenase lysate, and 4 U of lactate dehydrogenase lysate. The reaction was started by the addition of 100 μl of a solution containing 9 parts potassium xylonate (450.1 g/l) and 1 part potassium arabonate (51.7 g/l). The batch was incubated for a total of 48 hours at 30° C. with continuous shaking (1200 rpm, Eppendorf Thermomixer). The total volume of the batch was 500 μl.

For processing, 20 μl of the batch was combined with 180 μl of ultrapure water and 200 μl of MeOH and incubated at 60° C. for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For HPLC, 150 μl of the clear supernatant was transferred into HPLC vials for analysis (RI detection). The results are presented in the following table.

| | | | | conversion |
|---|---|---|---|---|
| | | concentration | mass | after 48 h |
| 100 g/l substrate | | [g/l] | [mg] | [%] |
| substrates | potassium xylonate | 90.0 | 45.0 | 52 * |
| (t = 0 h) | potassium | 10.3 | 5.2 | |
| | arabonate | | | |
| products | glycolate ** | 18.5 | 9.3 | 50 |
| (t = 48 h) | lactate ** | 18.4 | 9.2 | 42 |

* The conversion of the substrates may only be specified as a sum parameter due to peak overlap in HPAEC.
** The concentrations and masses of the products glycolate and lactate, as well as their conversions, were calculated for the respective free acids.

The table above indicates that the method is also suitable for the conversion of more concentrated substrate solutions.

### Example 5

Treatment of a Mixture of Glycolaldehyde and Pyruvate with Glycolaldehyde Dehydrogenase and/or Lactate Dehydrogenase and NAD⁺

In 2 ml Eppendorf vials, the following components were prepared: 100 μl of a 500 mM TEA-HCl buffer (pH 8.5; final concentration 100 mM), 0.1 U of lactate dehydrogenase (as lysate) and/or 0.1 U of glycolaldehyde dehydrogenase (as lysate), and a cofactor solution (NADH, NAD⁺). The final enzyme units and cofactor concentrations in the batches are listed in the table below. All batches were filled to a total volume of 400 μl with the appropriate amount of deionised water.

The reactions were started by the addition of 100 μl of a substrate solution (50 mM sodium pyruvate and 50 mM glycolaldehyde in deionised water). The batches were incubated for 1 hour at 30° C. and 1200 rpm in an Eppendorf Thermomixer. The total volume of each batch was 500 μl.

For processing, 200 μl of one of the batches was combined with 200 μl of MeOH and incubated at 60° C. for 20 minutes (1200 rpm, Eppendorf Thermomixer). Following denaturation, the samples were centrifuged for 10 minutes at maximum g-force. The clear supernatant was diluted 1:100 and analysed using HPAEC (conductivity detection) for the determination of pyruvate and lactate. For HPLC (RI detection), 150 μl of the clear supernatant was transferred into HPLC vials for analysis (determination of glycolate and glycolaldehyde*). The results are presented in the following table.

The concentrations of glycolaldehyde and pyruvate were 10 mM in all batches.

| | | | NADH | NAD⁺ | Pyruvate | Lactate | Glycolate |
|---|---|---|---|---|---|---|---|
| | Lactate- | Glycolaldehyde- | conc. | conc. | conc. | conc. | conc. |
| Batch | DH [U] | DH [U] | [mM] | [mM] | [mM] | [mM] | [mM] |
| 1 | 0.1 | 0.1 | 0 | 10 | 1.5 | 7.9 | 9.9 |
| 2 | 0.1 | 0.1 | 0 | 0.1 | 5.1 | 4.3 | 5.1 |
| 3 | 0 | 0.1 | 0 | 10 | 9.3 | 0.2 | 4.7 |
| 4 | 0.1 | 0 | 10 | 0 | 4.5 | 5.9 | 0 |

*The concentration of glycolaldehyde cannot be unequivocally determined due to the peak overlap of lactate and glycolaldehyde in the HPLC analysis.

The table indicates that when both lactate dehydrogenase and glycolaldehyde dehydrogenase are used simultaneously, the yields of glycolate and lactate (batch 1) are significantly increased at the same NAD⁺/NADH concentrations (10 mM) compared to batches 3 (glycolaldehyde dehydrogenase only) and 4 (lactate dehydrogenase only) (lactate: from 5.9 mM to 7.9 mM; glycolate: from 4.7 mM to 9.9 mM).

### LITERATURE

Sharad J. (2013). Glycolic acid peel therapy—a current review. Clinical, Cosmetic and Investigational Dermatology, 6, 281-288. https://doi.org/10.2147/CCID.S34029
Panova, A., Mersinger, L. J., Liu, Q., Foo, T., Roe, D. C., Spillan, W. L., Sigmund, A. E., Ben-Bassat, A., Wagner, L. W., O'Keefe, D., Wu, S., Petrillo, K., Payne, M. S., Breske, S. T., Gallagher, F. G. & DiCosimo, R. (2007). Chemoenzymatic Synthesis of Glycolic Acid. Advanced Synthesis & Catalysis, 349(8-9), 1462-1474. https://doi.org/10.1002/adsc.200700061
Ben-Bassat, A., Walls, A. M., Plummer, M. A., Sigmund, A. E., Spillan, W. & DiCosimo, R. (2008). Optimization of Biocatalyst Specific Activity for Glycolic Acid Production. Advanced Synthesis & Catalysis, 350(11-12), 1761-1769. https://doi.org/10.1002/adsc.200800228
Gao, X., Ma, Z., Yang, L. & Ma, J. (2014). Enhanced Bioconversion of Ethylene Glycol to Glycolic Acid by a Newly Isolated Burkholderia sp. EG13. Applied Biochemistry and Biotechnology, 174, 1572-1580. https://doi.org/10.1007/s12010-014-1114-9
Dapsens, P. Y., Mondelli, C., Kusema, B. T., Verel, R. & Pérez-Ramírez, J. (2014). A continuous process for glyoxal valorisation using tailored Lewis-acid zeolite catalysts. Green Chemistry, 16(3), 1176-1186. https://doi.org/10.1039/C3GC42353K
Hromádková, Z. & Ebringerová, A. (1995). Isolation and Characterization of Hemicelluloses of Corn Hulls. Chemical Papers 49(2), 97-101. https://chempap.org/?id=7&paper=3556
Collins, S. R. A., Wellner, N., Martinez Bordonado, I., Harper, A. L., Miller, C. N., Bancroft, I. & Waldron, K. W. (2014). Variation in the chemical composition of wheat straw: the role of tissue ratio and composition. Biotechnology for Biofuels, 7, 121. https://doi.org/10.1186/s13068-014-0121-y
Claassens, N. J., Burgener, S., Vögeli, B., Erb, T. J. & Bar-Even, A. (2019). A critical comparison of cellular and cell-free bioproduction systems. Current Opinion in Biotechnology, 60, 221-229. https://doi.org/10.1016/j.copbio.2019.05.003
Salusjärvi, L., Havukainen, S., Koivistoinen, O. & Toivari, M. (2019). Biotechnological production of glycolic acid and ethylene glycol: current state and perspectives. Applied Microbiology and Biotechnology, 103, 2525-2535. https://doi.org/10.1007/s00253-019-09640-2
Pereira, B., Li, Z.-J., De Mey, M., Lim, C. G., Zhang, H., Hoeltgen, C. & Stephanopoulos G. (2016). Efficient utilization of pentoses for bioproduction of the renewable two-carbon compounds ethylene glycol and glycolate. Metabolic Engineering, 34, 80-87. https://doi.org/10.1016/j.ymben.2015.12.004
Deng, Y., Ma, N., Zhu, K., Mao, Y., Wei, X. & Zhao, Y. (2018). Balancing the carbon flux distributions between the TCA cycle and glyoxylate shunt to produce glycolate at high yield and titer in Escherichia coli. Metabolic Engineering, 46, 28-36. https://doi.org/10.1016/j.ymben.2018.02.008
Alkim, C., Trichez, D., Cam, Y., Spina, L., François, J. M. & Walther, T. (2016). The synthetic xylulose-1 phosphate pathway increases production of glycolic acid from xylose-rich sugar mixtures. Biotechnology for Fuels, 9, 201. https://doi.org/10.1186/s13068-016-0610-2
Salusjärvi, L., Toivari, M., Vehkomäki, M.-L. Koivistoinen, O., Mojzita, D., Niemelä, K., Penttilä, M. & Ruohonen, L. (2017). Production of ethylene glycol or glycolic acid from D-xylose in Saccharomyces cerevisiae. Applied Microbiology and Biotechnology, 101, 8151-8163. https://doi.org/10.1007/s00253-017-8547-3
Uranukul, B., Woolston, B. M., Fink, G. R. & Stephanopoulos, G. (2018). Biosynthesis of monoethylene glycol in Saccharomyces cerevisiae utilizing native glycolytic enzymes. Metabolic Engineering, 51, 20-31. https://doi.org/10.1016/j.ymben.2018.09.012
Buchner, E. 1897. Alkoholische Gärung ohne Hefezellen. Berichte der deutschen chemischen Gesellschaft, 30:1110-1113. https://doi.org/10.1002/cber.19010340231
Welch, P. & Scopes, R. K. (1985). Studies on cell-free metabolism: Ethanol production by a yeast glycolytic system reconstituted from purified enzymes. Journal of Biotechnology, 2(5), 257-273. https://doi.org/10.1016/0168-1656(85)90029-X
Goldberg, K., Schroer, K., Lütz, S., & Liese, A. (2007). Biocatalytic ketone reduction—a powerful tool for the production of chiral alcohols—part I: processes with isolated enzymes. Applied Microbiology and Biotechnology, 76, 237-248. https://doi.org/10.1007/s00253-007-1002-0
Schrittwieser, J. H., Velikogne, S., Hall, M. & Kroutil, W. (2018). Artificial Biocatalytic Linear Cascades for Preparation of Organic Molecules. Chemical Reviews, 118(1), 270-348. https://doi.org/10.1021/acs.chemrev.7b00033
Dahms, A. S. (1974). 3-Deoxy-D-pentulosonic acid aldolase and its role in a new pathway of D-xylose degradation. Biochemical and Biophysical Research Communications, 60(4), 1433-1439. https://doi.org/10.1016/0006-291X(74)90358-1
Kopp, D., Bergquist, P. L., & Sunna, A. (2020). Enzymology of Alternative Carbohydrate Catabolic Pathways. Catalysts, 10(11), 1231. https://doi.org/10.3390/catal10111231
Wee, Y.-J., Kim, J.-N., & Ryu, H.-W. (2006). Biotechnological Production of Lactic Acid and Its Recent Applications. Food Technology & Biotechnology, 44(2), 163-172. https://www.ftb.com.hr/80-volume-44-issue-no-2/445-biotechnological-production-of-lactic-acid-and-its-recent-applications
Smith, W. P. (1996). Epidermal and dermal effects of topical lactic acid. Journal of the American Academy of Dermatology, 35(3), 388-391. https://doi.org/10.1016/s0190-9622(96) 90602-7
Balla, E., Daniilidis, V., Karlioti, G., Kalamas, T., Stefanidou, M., Bikiaris, N. D., Vlachopoulos, A., Koumentakou, I. & Bikiaris, D. N. (2021). Poly(lactic Acid): A Versatile Biobased Polymer for the Future with Multifunctional Properties—From Monomer Synthesis, Polymerization Techniques and Molecular Weight Increase to PLA Applications. Polymers, 13(11), 1822. https://doi.org/10.3390/polym13111822
Boer, H., Andberg, M., Pylkkänen, R., Maaheimo, H. & Koivula, A. (2019). In vitro reconstitution and characterisation of the oxidative D-xylose pathway for production of organic acids and alcohols. AMB Express, 9, 48. https://doi.org/10.1186/s13568-019-0768-7
Lin, B. & Tao, Y. (2017). Whole-cell biocatalysts by design. Microbial Cell Factories, 16, 106. https://doi.org/10.1186/s12934-017-0724-7
Moore, S. & Link, K. P. (1940). CARBOHYDRATE CHARACTERIZATION: I. THE OXIDATION OF ALDOSES BY HYPOIODITE IN METHANOL II. THE IDENTIFICATION OF SEVEN ALDO-MONOSACCHARIDES AS BENZIMIDAZOLE DERIVATIVES. Journal of Biological Chemistry, 133, 293-311. https://doi.org/10.1016/S0021-9258(18)73312-7
Setubal, J. C., dos Santos, P., Goldman, B. S., Ertesvåg, H., Espin, G., Rubio, L. M., Valla, S., Almeida, N. F., Balasubramanian, D., Cromes, L., Curatti, L., Du, Z., Godsy, E., Goodner, B., Hellner-Burris, K., Hernandez, J. A., Houmiel, K., Imperial, J., Kennedy, C., Larson, T. J., Latreille, P., Ligon, L. S., Lu, J., Mærk, M., Miller, N. M., Norton, S., O'Carroll, I. P., Paulsen, I., Raulfs, E. C., Roemer, R., Rosser, J., Segura, D., Slater, S., Stricklin, S. L., Studholme, D. J., Sun, J., Viana, C. J., Wallin, E., Wang, B., Wheeler, C., Zhu, H., Dean, D. R., Dixon, R. & Wood, D. (2009). Genome sequence of Azotobacter vinelandii, an obligate aerobe specialized to support diverse anaerobic metabolic processes. Journal of Bacteriology, 191(14), 4534-4545. https://doi.org/10.1128/JB.00504-09
Protein [Internet]. Bethesda (MD): National Library of Medicine (US), National Center for Biotechnology Information. Accession No. ACO81022.1, L-arabonate dehydratase [Azotobacter vinelandii DJ]; [accessed on 04.10.2022]. Obtainable at: https://www.ncbi.nlm.nih.gov/protein/226721851
Wolterink-van Loo, S., Siemerink, M. A. J., Perrakis, G., Kaper, T., Kengen, S. W. M. & van der Oost, J. (2009). Improving low-temperature activity of Sulfolobus acidocaldarius 2-keto-3-deoxygluconate aldolase. Archaea, 2(4), 233-239. https://doi.org/10.1155/2009/194186
Hidalgo, E., Chen, Y.-M., Lin, E. C. C. & Aguilar, J. (1991). Molecular cloning and DNA sequencing of the Escherichia coli K-12 ald gene encoding aldehyde dehydrogenase. Journal of Bacteriology, 173(19), 6118-6123. https://doi.org/10.1128/jb.173.19.6118-6123.1991
Zheng, Y., Guo, S., Guo, Z. & Wang, X. (2004). Effects of N-terminal deletion mutation on rabbit muscle lactate dehydrogenase. Biochemistry (Moscow), 69(4), 401-406. https://doi.org/10.1023/b:biry.0000026195.31821.e1

1. A method for producing an aqueous solution containing an alkali salt of glycolic acid and lactic acid, by treating an alkali salt of xylonic acid and/or arabonic acid in an aqueous solution in vitro with an enzyme system comprising a dehydratase, an aldolase, a glycolaldehyde dehydrogenase, a lactate dehydrogenase, and NAD⁺ as a cofactor, after which the enzyme system is separated.

2. The method according to claim 1, wherein the concentration of the alkali salt of xylonic acid and/or arabonic acid in the aqueous solution is between 50 and 300 g/l.

3. The method according to claim 2, wherein the concentration of the alkali salt of xylonic acid and/or arabonic acid in the aqueous solution is between 100 and 200 g/l.

4. The method according to claim 3, wherein the alkali salt of xylonic acid and/or arabonic acid in the aqueous solution is between 150 and 250 g/l.

5. The method according to claim 1, wherein the method is carried out at a temperature between 2° and 50° C.

6. The method according to claim 5, wherein the method is carried out at a temperature between 25 and 40° C.

7. The method according to claim 6, wherein the method is carried out at a temperature between 3° and 40° C.

8. The method according to claim 1, wherein the enzymes forming the enzyme system are present as a lysate of the corresponding cells forming them.

9. The method according to claim 1, wherein the enzymes forming the enzyme system are present as a homogenate of the corresponding cells forming them.

10. The method according to claim 1, wherein the enzymes forming the enzyme system are present as a suspension of the corresponding cells forming them.

11. The method according to claim 10, wherein the pH is between 7.5 and 8.5.
